(19) United States
(12) Patent Application Publication
(10) Pub. No.: US 2006/0122119 A1
(43) Pub. Date: Jun. 8, 2006

(54) Peptides for use in antitumor immunotherapy

(75) Inventors: Boris Linard, Saint-Herblain (FR); Francine Jotereau, Nantes (FR); Houssem Benlalam, L'Hay les Roses (FR); Elizabeth Diez, Saint Fiacre Sur Maine (FR); Yannick Guilloux, Nantes (FR); Nathalie Labarriere, Nantes (FR); Nadine Gervois, Nantes (FR); Laurent Derre, Nantes (FR)

Correspondence Address: OBLON, SPIVAK, MCCLELLAND, MAIER & NEUSTADT, P.C., 1940 DUKE STREET, ALEXANDRIA, VA, 22314, US

(73) Assignees: Institut Nat'l De La Sante Et De La Recherche Med., Paris (FR); Universite De Nantes, Nantes (FR)

(21) Appl. No.: 10/506,334

(22) PCT Filed: Mar. 4, 2003
(86) PCT No.: PCT/FR03/00698

§ 371 Date: May 25, 2005

(30) Foreign Application Priority Data
Mar. 4, 2002 (FR) 02/02703

(51) Int. Cl. A61K 38/10

(52) U.S. Cl. 514/14; 514/15

(57) ABSTRACT

The invention concerns the use of immunogenic peptides representing T epitopes presented by the MHC I, derived from Melan-A, MAGE-A6, gp 100, tyrosinase and NY-ESO-1 antigens for the diagnosis or treatment of melanomas in HLA-B35 subjects.

The present invention relates to peptides representing epitopes shared by tumor antigens, and to their use in immunotherapy.

Immunization or peptide immunotherapy is a therapeutic approach which is currently the subject of great interest for the prevention or treatment of cancers. Its principle is based on immunization with peptides which reproduce T epitopes of tumor-associated antigens (TAAs) recognized by cytotoxic T lymphocytes (CTLs), which play a major role in eliminating the cancer cells expressing these antigens at their surface.

It will be recalled that CTLs do not recognize whole protein antigens, but peptide fragments thereof, represented by the molecules of the major histocompatibility complex (MHC) expressed at the surface of various cells. It is these peptide fragments which constitute the T epitopes. The peptides presented by the major histocompatibility class I (MHC I) complex generally have 8 to 11 amino acids, and are recognized by CD8+ T cells, which represent the major component of the cytotoxic response. The peptides presented by the major histocompatibility complex class II (MHC II) generally have 13 to 18 amino acids and are recognized by CD4+ T cells.

The identification of these epitopes, and in particular of those presented by MHC I (given the essential role of the CD8+ response in cytotoxicity), therefore constitutes an essential step for developing antitumor immunotherapy compositions.

In the case of melanomas, two main classes of melanoma-associated antigens (MAAs) have been identified: the melanoma specific antigens, which are expressed little or not at all in normal tissues, and the melanocyte differentiation antigens, which are also expressed by melanocytes (for review, see CASTELLI et al., 2000, J Cell Physiol, 182, 323-31; KIRKIN et al., 2002, Cancer Invest, 20, 222-36).

The melanocyte differentiation antigens, such as Melan-A/MART-1, gp-100 and tyrosinase, are expressed by a considerable proportion of melanoma-type tumors. In addition, these antigens are effectively recognized both by CTLs from normal individuals and by those from patients suffering from melanoma (BENLALAM et al., 2001, Eur J Immunol, 31, 2007-15; KAWAKAMI et al., 2000, J Immunother, 23, 17-27; LABARRIERE et al., 1998, Int J Cancer, 78, 209-15; PITTET et al., 1999, J Exp Med, 190, 705-15; VALMORI et al., 2002, Cancer Res, 62, 1743-50). At the current time, more than thirty epitopes of these antigens recognized by CTLs are known, close to two thirds of which are present in the HLA-A*0201 context.

The melanoma-specific antigens include families of antigens referred to as “cancer-testes shared antigens”: MAGE, GAGE, BAGE and LAGE. These antigens, which are expressed by various tumors, generate CTL epitopes presented in a large variety of HLA contexts, including HLA-B and HLA-C (KIRKIN et al., 2002). With the exception of NY-ESO-1 (JAGER et al., 1998, J Exp Med, 187, 265-70) and unlike the melanocyte differentiation antigens, cancer-testes shared antigens are rarely recognized by tumor-infiltrating lymphocytes (TILs). Most of the epitopes present in these antigens are identified by means of CTLs generated ex vivo by repeated stimulation of peripheral blood lymphocytes (PBLs) by antigen-presenting cells (APCs) loaded with antigens (SCHULTZ et al., 2001, Tissue Antigens, 57, 103-9), with the exception of the MAGE-A6 and MAGE-A12 epitopes, since they were identified with CTL clones derived from TILs (PANELLI et al., 2000, J Immunol, 164, 4382-92; ZOON & HERCEND, 1999, Eur J Immunol, 29, 602-7).

To date, most of the peptide immunotherapy trials on melanomas have been carried out in the HLA-A*0201 or HLA-A*0101 context and using only a limited number of TAA epitopes (LAU et al., 2001, J Immunother, 24, 66-78; MACKENSEN et al., 2000, Int J Cancer, 86, 385-92; MARCHAND et al., 1999, Int J Cancer, 80, 219-30; PANELLI et al., 2000, J Immunother, 23, 487-98; ROSENBERG et al., 1998, J Natl Cancer Inst, 90, 1894-900; SALGALLER et al., 1996, Cancer Res, 56, 4749-57). Now, the underexpression of HLAs, and the appearance of TAA variants having lost the epitopes recognized by CTLs, could constitute mechanisms allowing tumor cells to evade the immune system (FERRONE & MARINCOLA, 1995, Immunol Today, 16, 487-94; MAEURER et al., 1996, Clin Cancer Res, 2, 641-52), which could explain the poor efficacy of vaccines using peptides.

To overcome this drawback, it has been proposed to use polyvalent vaccines made up of a multitude of peptides derived from various TAAs and presented in varied HLA contexts. However, this requires the identification of new peptide/HLA complexes which are effectively presented by the tumor cells.

In addition, the identification of peptides presented in various HLA contexts also makes it possible to develop tools for measuring the T-cell response in the immunized patients. In fact, while the identification of novel antigenic peptides is essential for increasing the availability and the efficacy of vaccines, this is also the case for improving the monitoring of the CTL response of patients who have been immunized with peptides or other forms of antigens, such as whole recombinant proteins or recombinant viruses.

Insofar as HLA-B35 constitutes one of the most common HLA-B alleles, present in 20% of the Caucasian population (60% of which corresponds to the B*3501 allele; MORI et al., 1997, Transplantation, 64, 1017-27), the identification of novel antigenic peptides presented in the HLA-B35 context is very desirable for the development of cancer immunotherapy.

The inventors have identified novel epitopes which are derived from melanocyte antigens or from cancer-testes shared antigens, and which are presented in the HLA-B35 context.

The peptides which reproduce these epitopes can be used for a diagnostic or therapeutic purpose, in the context of the prevention or treatment of melanomas in patients expressing an HLA-B35 allele, in particular HLA-B*3501 or HLA-B*3503.

A subject of the present invention is the use of at least one immunogenic peptide representing a T epitope presented by MHC I, chosen from:

a) a peptide comprising the sequence EX₁AGIGILX₂ (SEQ ID NO: 1) in which X₁ represents A or P, and X₂ represents T or Y, capable of inducing a cytotoxic response directed against the Melan-A antigen;

b) a peptide comprising the sequence EVDPIGHVY (SEQ ID NO: 2), capable of inducing a cytotoxic T response directed against the MAGE-A6 antigen;

c) a peptide comprising the sequence VPLDCVLYR (SEQ ID NO: 3), capable of inducing a cytotoxic response directed against the gp100 antigen;

d) a peptide comprising the sequence TPRLPSSADVEF (SEQ ID NO: 4), capable of inducing a cytotoxic response directed against the tyrosinase antigen;

e) a peptide comprising the sequence MPFATPMEA (SEQ ID NO: 5), capable of inducing a cytotoxic response directed against the NY-ESO-1 antigen; for obtaining a medicinal product intended for antitumor immunotherapy in an HLA-B35 patient. Advantageously, said medicinal product is intended for the treatment of melanomas.

Advantageously, use may be made of:

at least one peptide a) corresponding to a sequence chosen from: TAEEAAGIGILTV (SEQ ID NO: 6), EAAGIGILTVIL (SEQ ID NO: 7), EAAGIGILTV (SEQ ID NO: 8), EAAGIGILTY (SEQ ID NO: 9), EAAGIGILY (SEQ ID NO: 10), EPAGFIGILTY (SEQ ID NO: 11), EPAGIGILTV (SEQ ID NO: 12);

a peptide b) corresponding to the sequence EVDPIGHVY (SEQ ID NO: 2);

at least one peptide c) corresponding to a sequence chosen from VPLDCVLYR (SEQ ID NO: 3) and VPLDCVLYRY (SEQ ID NO: 13);

at least one peptide d) corresponding to a sequence chosen from TPRLPSSADVEFCL (SEQ ID NO: 14) and TPRLPSSADVEF (SEQ ID NO: 4);

at least one peptide e) corresponding to a sequence chosen from: LAMPFATPMEAEL (SEQ ID NO: 15), LAMPFATPMEAE (SEQ ID NO: 16), MPFATPMEAEL (SEQ ID NO 17), MPFATPMEAE (SEQ ID NO: 18) and MPFATPMEA (SEQ ID NO: 5).

According to a preferred embodiment of the invention, use is made of a combination comprising at least two peptides of two different categories among the categories a), b), c), d) and e) defined above, in order to able to induce a cytotoxic response against at least two tumor antigens.

A subject of the present invention is also a multiepitope composition combining at least two peptides of two different categories among the categories a), b), c), d) and e) above. Advantageously, these compositions comprise at least one peptide of each of these categories a), b), c), d) and e).

Multiepitope compositions in accordance with the invention may also comprise one or more other immunogenic peptide(s) derived from the antigens mentioned above, or from different antigens. These peptides may represent epitopes derived from the same antigen, or from two or more different antigens. By way of example, mention will be made of the peptide EVDPIGHLY (SEQ ID NO: 19), which represents an epitope of MAGE-A3, already known to be capable of being presented in an HLA-B35 context (PCT application WO 01/53833).

These compositions may also comprise, in order to be more widely usable on a population in which the individuals carry different HLA alleles, one or more peptides presented by MHC I molecules other than HLA-B35. By way of example, mention will be made of the peptide PLDCVLYRY (SEQ ID NO: 20), described hereinafter.

Multiepitope compositions in accordance with the invention may in particular be in the form of a chimeric polypeptide comprising one or more copies of each of the chosen epitopes.

Chimeric polypeptides in accordance with the invention can be readily obtained by methods known in themselves, and in particular by conventional recombinant DNA techniques.

The subject of the present invention is also a polynucleotide encoding a chimeric polypeptide in accordance with the invention, and also a nucleic acid vector containing said polynucleotide.

The present invention also encompasses the use of said polynucleotide or said nucleic acid vector in antitumor immunotherapy.

Nonlimiting examples of implementation of the present invention are given hereinafter.

It is possible, for example, to inject a patient to be treated with a peptide, a chimeric polypeptide or a multiepitope composition as defined above, optionally combined with a suitable adjuvant.

It is also possible to use one of the peptides defined above, for loading professional HLA-B35 antigen-presenting cells in vitro, in particular dendritic cells, in order to induce the proliferation of antitumor CTLs, as described, for example, by BAKKER et al. (Cancer Res., 55, 5330-5334, 1995) or VAN ELSAS et al. (Eur. J. Immunol., 26, 1683-1689, 1996).

The HLA-B35 antigen-presenting cells loaded in this way are also part of the subject of the present invention.

The polynucleotides in accordance with the invention preferably integrated into nucleic acid vectors, in particular viral vectors such as adenoviruses, can also be administered to the patient to be treated by injection.

A polynucleotide comprising a sequence encoding a peptide defined by one of the sequences SEQ ID NO: 1 to 18 above, and in particular a polynucleotide encoding a chimeric polypeptide according to the invention, can also be used for transfecting professional HLA-B35 antigen-presenting cells in vitro, in particular dendritic cells, which are then injected into the patient, as described, for example, by KAPLAN et al., (J. Immunol., 163(2), 699-707, 1999) or KIM et al. (Annals of Surgical Oncology, 5(1), 64-76, 1998). These transfected antigen-presenting cells are also part of the subject of the invention.

The present invention also encompasses the use of the peptides defined above, for detecting in vitro, CTLs directed against one or more of the antigens Melan-A, MAGE-A6, gp100, tyrosinase and NY-ESO-1, in a biological sample obtained from an HLA-B35 individual.

These peptides can also be used for performing specific sorting of these CTLs. The CTLs thus isolated can then be amplified in vitro and reinjected in a large number (of the order of a billion) into the patient.

A subject of the present invention is also therapeutic compositions comprising, as active principle, a mutated ras peptide, a multiepitope composition, a chimeric polypeptide, a polynucleotide, or an antigen-presenting cell in accordance with the invention.

The therapeutic compositions in accordance with the invention can also comprise the usual excipients, and also adjuvants conventionally used in immunotherapy and which make it possible, for example, to promote the administration of the active principle, to stabilize it, to increase its immunogenicity, etc.

Among the peptides of the categories a), b), c), d) and e) mentioned above, some were already known to be capable of inducing a CTL response, but in contexts other than HLA-B35.

Others had never been described as epitopes capable of inducing a cytotoxic T response and had therefore never been isolated. They are the following peptides:

the peptides EAAGIGILTY (SEQ ID NO: 9), EAAGIGILY (SEQ ID NO: 10), EPAGIGILTY (SEQ ID NO: 11), EPAGIGILTV (SEQ ID NO: 12);

the peptides VPLDCVLYR (SEQ ID NO: 3) and VPLDCVLYRY (SEQ ID NO: 13);

the peptides TPRLPSSADVEFCL (SEQ ID NO: 14) and TPRLPSSADVEF (SEQ ID NO: 4).

The present invention also encompasses these particular peptides, and also any multiepitope composition comprising at least one of these peptides. This includes in particular chimeric polypeptides containing at least one of these peptides. The polynucleotides encoding these chimeric polypeptides and the nucleic acid vectors containing these polynucleotides are also part of the subject of the present invention.

The inventors have also noted that the peptide VPLDCVLYRY (SEQ ID NO: 13) derived from the gp100 antigen is also recognized, in the HLA*A0101 context, by a CD8 T clone (M199.6.12) derived from a population of melanoma TILs. From this peptide, they have identified a shorter peptide (PLDCVLYRY, SEQ ID NO: 20) which is not recognized in the HLA-B35 context but is very effectively recognized in the A*0101 context. The peptide SEQ ID NO: 20 is also part of the subject of the present invention, as are the chimeric polypeptides containing at least this peptide. The polynucleotides encoding these chimeric polypeptides, and the nucleic acid vectors containing these polynucleotides, are also part of the subject of the present invention. This peptide, the chimeric polypeptide containing it, and also the polynucleotides encoding these chimeric polypeptides, can be used in the context of the detection or treatment of melanomas in HLA-A1, and in particular HLA*A0101 individuals, according to the same techniques as those indicated above for the peptides recognized in the HLA-B35 context.

Furthermore, studies carried out on animal models have made it possible to identify other tumor rejection antigens, a large number of which are mutated oncogenic proteins (PREHN et al., J. Natl. Cancer Inst., 18, 769, 1998; DE PLAEN et al., PNAS, 85, 2274, 1988; DUBEY et al., J. Exp. Med., 185, 695, 1997). The presentation, by MHC molecules, of the mutated fragments of these proteins at the surface of the tumor cells induces the specific destruction of said cells by CTLs and rejection of the tumor. In humans, the mutated oncogenic proteins also appear to be particularly advantageous targets for antitumor immunotherapy. However, this involves identifying the epitopes that are present at the surface of the tumor cells and capable of inducing a T cytotoxic response.

Among the oncogenes most commonly involved in various types of tumors, mention may be made of the ras oncogenes (K-ras, H-ras and N-ras) which result from point mutations (substitution of a single amino acid) of the ras p21 protooncogenes. These mutations occur essentially at codon 12, at codon 13 or at codon 61 (BOS, Cancer Res., 49, 4682, 1989; WEIJZEN et al., Leukemia, 13, 502, 1999). Due to the limited number of oncogenic substitutions which may occur, ras mutations present in many identical tumors can thus generate shared tumor epitopes, presented by a significant fraction of human tumors (WEIJZEN et al., Leukemia, 13, 502, 1999).

It has thus been proposed to use synthetic peptides which reproduce mutated ras epitopes, in antitumor immunotherapy. Thus, PCT application WO 92/14756 proposes the use of peptides which reproduce ras epitopes mutated at codon 12 or at codon 61. However, these epitopes are presented by MHC II (DQ and DR) and therefore induce a CD4+ response. Now, where it may be advantageous to induce this type of response, insofar as CD4+ helper lymphocytes make it possible to increase the cytotoxic response (WALTER et al., N. Engl. J. Medicine, 333, 1038, 1995), the CD8+ response remains the essential participant in the cytotoxicity. In addition, recent studies carried out in mice suggest that immunization with peptides presented by MHC II could induce stimulation of tumor growth instead of the protection hoped for (SIEGEL et al., J. Exp. Med., 191, 1945, 2000).

Several ras epitopes mutated at positions 12, 13 or 61 have been selected on the basis of their ability to anchor to MHC class I molecules (VAN ELSAS et al., Int. J. Cancer, 61, 389, 1995; BERGMANN et al., Cell Immunol., 187, 103, 1998; GOUTTEFANGEAS et al., Human Immunol., 55, 117, 1997). The peptides thus selected can stimulate the growth of specific CTLs using PBLs in vitro. However, these CTLs recognize the mutated tumor cells weakly, suggesting that the endogenous expression of these epitopes is limited (VAN ELSAS et al., Int. J. Cancer, 61, 389, 1995; ABRAMS et al., Cell Immunol., 182, 137, 1997; BERGMANN et al., Cell Immunol. 187, 103, 1998), and that they do not therefore allow the efficient elimination of the tumor cells by means of the specific CTLs, which considerably limits their value in immunotherapy.

It therefore appears to be necessary to have other mutated ras epitopes that are MHC I restricted and effectively presented by a significant fraction of human tumors.

The inventors have now identified a ras epitope which is mutated at position 61 by substitution of a glutamine with an arginine (Q61R), and is MHC I-restricted. This epitope, hereinafter referred to as 55-64^{Q61R} is effectively presented by several HLA-A*0101+ melanoma lines expressing a ras oncogene carrying the Q61R mutation. It is capable of specifically inducing the expansion of tumor-infiltrating lymphocyte (TIL) clones obtained from these melanomas. In addition, the dendritic cells loaded with this peptide effectively stimulate specific CTLs using peripheral blood lymphocytes (PBLs) from normal HLA-A*0101 donors, and these CTLs recognize all the HLA-A*0101 melanoma lines expressing the Q61R ras oncogene, and do not recognize cells expressing the non-mutated ras protein.

The peptide 55-64^{Q61R} does not have an anchoring capacity greater than that of the corresponding wild-type peptide. The HLA-A*0101-binding affinity of the wild-type peptide is similar to that of the peptide 55-64^{Q61R}. However, even at high concentrations, this wild-type peptide can neither induce the expansion of specific TILs, nor that of specific CTLs. It therefore appears that the substitution of a glutamine with an arginine at position 61 creates a novel epitope presented by HLA-A*0101.

In addition, analysis carried out on the BIMAS databank (http://bimas.dcrt.nih.gov/molbio/hla bind; PARKER et al., J. Immunol. 152, 163, 1994), shows that this peptide has a binding score that is identical for HLA-A*0101 and HLA-B*1501. Consequently, a subject of the present invention is also the use of an immunogenic mutated ras peptide of sequence ILDTAGREEY (SEQ ID NO: 35) for obtaining an medicinal product intended for the immunotherapy-based treatment of tumors in an HLA-A*0101 or HLA-B*1501 patient.

Advantageously, said medicinal product can be used for the treatment of tumors expressing a K-ras, H-ras or N-ras protein mutated by substitution of the glutamine at position 61 with an arginine.

Mention will in particular be made of melanomas, and also other tumors in which the ras mutations affecting residue 61 are detected with high frequency, such as congenital melanocytic naevi (PAPP et al., Journal of Medical Genetics, 36, 610, 1999), multiple myelomas (BEZIEAU et al. Hum. Mutat., 18, 281, 2001) and thyroid tumors (ESAPA et al., Clinical Endocrinology, 50, 529, 1999).

Said peptide can be used especially for multiepitope compositions, and in particular for chimeric polypeptides, as mentioned above. A polynucleotide encoding such a chimeric polypeptide and also a nucleic acid vector containing said polynucleotide can also be used as mentioned above.

Said peptide or said polynucleotide can also be used, respectively, for loading or transfecting, in vitro, professional HLA-A*0101 or HLA-B1501 antigen-presenting cells, in order to induce the proliferation of antitumor CTLs.

The HLA-A*0101 or HLA-B*1501 antigen-presenting cells loaded or transfected in this way are also part of the subject of the present invention.

Said mutated ras peptide can also be used for detecting, in vitro, CTLs directed against the mutated ras antigen from which it is derived, in a biological sample obtained from an HLA-A*0101 or HLA-B*1501 individual. It can also be used for performing the specific sorting of these CTLs.

The present invention will be understood more fully from the further description which follows, which refers to nonlimiting examples which illustrate the properties of mutated ras peptides in accordance with the invention which can be used in immunotherapy, by implementing the method in accordance with the invention.

### EXAMPLE 1

### Demonstration of Antigenic Epitopes Presented in the HLA-B35 Context and Recognized by CTL Clones

In a previous study (BENLALAM et al., 2001), the inventors obtained HLA-B35-restricted CD8+ TIL clones recognizing, respectively, the tyrosinase (TIL M171), Melan-A (TIL M171 and M28) and gp100 (TIL M171, M28 and M110) antigens.

They completed this collection of clones with a clone recognizing MAGE-A3/MAGE-A6(TIL-M171) and a clone recognizing NH-ESO-1 (TIL M118).

The response of these various TIL clones with respect to COS-7 cells cotransfected with the relevant antigen and HLA-B35 allele is detected by measuring their TNF production.

The COS-7 cells, cultured in DMEM medium (Sigma) containing 10% of fetal calf serum, antibiotics and L-glutamine, were transfected with the cDNA encoding one of the alleles HLA-B*3501, HLA-A*3503, HLA-B*3508, alone or in combination with a cDNA encoding one of the antigens MAGE-A3, MAGE-A6, tyrosinase, Melan-A/MART-1, gp-100 and NY-ESO1/LAGE-2. The transfection was carried out according to the protocol described by DE PLAEN et al. (Methods, 12, 125-42, 1997). 16.5 10³ COS-7 cells were transfected with 100 ng of plasmid pcDNA3 (Invitrogen, San Diego, Calif.) containing the cDNA of the HLA-B*35 allele concerned and, for the cotransfection, 100 ng of plasmid containing the cDNA encoding the chosen antigen.

These COS-7 cells were used, 48 h after transfection, to stimulate the various TIL clones (2×10³ to 104). The culture supernatants were removed 6 hours later and the TNF concentration thereof was determined by measuring the cytotoxicity of these culture supernatants for the clone 13 WEHI 164, as described by DE PLAEN et al. (1997, mentioned above).

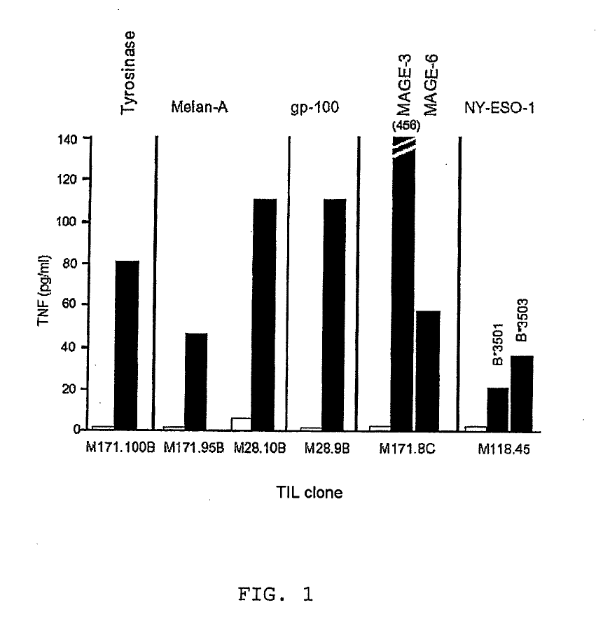

The results are given in FIG. 1.

On the y-axis, the concentration of TNF in pg/ml

On the x-axis, the various TIL populations.

□: Secretion of TNF by the TIL clones in the presence of the COS-7 cells transfected only with a cDNA encoding an HLA-B35 molecule.

▪: Secretion of TNF by the TIL clones in the presence of the COS-7 cells cotransfected with a cDNA encoding an HLA-B35 molecule and a cDNA encoding a MAA.

Two clones (M171.95B and M28.10B) recognize a Melan-A/B*3501 complex, one clone (M28.9B) recognizes a gp100/B*3501 complex, one clone (M171.100B) recognizes a tyrosinase/B*3501 complex, one clone (M171.8C) recognizes a MAGE-A3 and MAGE-A6 epitope in the context of B*3501, and one clone (M118.45) recognizes an NY-ESO-1 epitope in the B*3501 and B*3503 contexts.

### EXAMPLE 2

### Identification of the Antigenic Epitopes Presented in the HLA-B35 Context

To identify the regions encoding the epitopes recognized by the TIL clones, the inventors constructed a series of fragments of the cDNAs of the various MAAs.

The Melan-A/MART-1 and NY-ESO-1 cDNA fragments were obtained by digestion with exonuclease III: the plasmids comprising the cDNA encoding Melan-A/MART-1 or NY-ESO-1 were opened with XbaI and ApaI, or SPHI and NotI, respectively. The fragments obtained were then digested with exonuclease III using the Erase a-base system (Promega, Madison, Wis.).

A restriction fragment of gp-100 (1156-1986) containing the nucleotides located between the KpnI site 1156 bp downstream of the initiation codon, and the end of the coding sequence was generated by enzymatic digestion with KpnI.

The cDNA fragments corresponding to the fragments of the tyrosinase and gp100 antigens were obtained by PCR from the plasmids containing the complete sequence encoding each of these two antigens.

The expression of these various fragments by COS-7 cells is carried out as in example 1. The responses of the various TIL clones against these COS-7 cells cotransfected with the relevant antigen fragments and HLA-B35 allele are measured as in example 1.

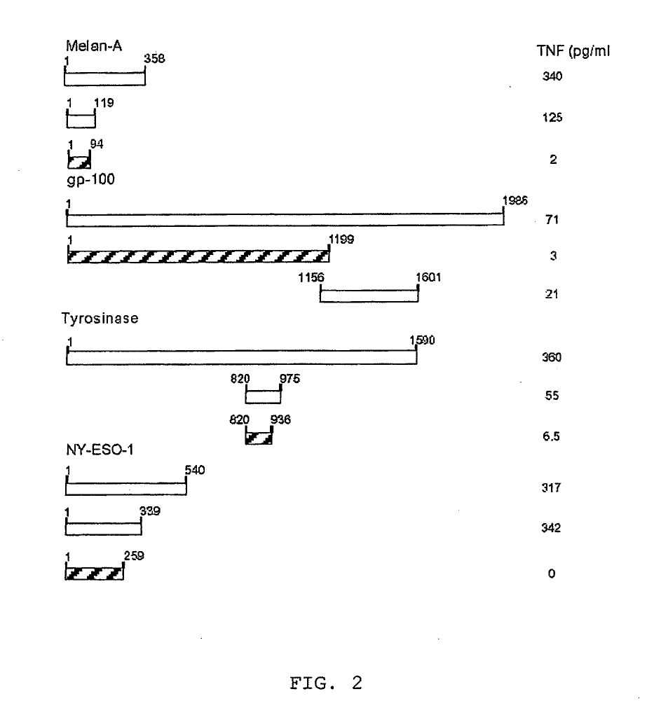

The results are given in FIG. 2:

The positions of the regions of the cDNAs encoding the potential epitopes are indicated in base pairs.

□: Fragment recognized by specific CTL clones (significant secretion of TNF).

[symbol]

: Fragment not recognized by specific CTL clones (non-significant secretion of TNF).

It appears that the various TIL populations recognize epitopes which are encoded, respectively, by the Melan-A cDNA fragment extending from nucleotides 95 to 119 (amino acids 32 to 39), the gp100 cDNA fragment 1200 to 1601 (amino acids 400 to 533), the tyrosinase cDNA fragment 937 to 975 (amino acids 313 to 325) and the NY-ESO-1 cDNA fragment 259 to 339 (amino acids 87 to 113).

### EXAMPLE 3

### Identification of Antigenic Peptides Presented in the HLA-B35 Context

The wild-type and modified peptides, the sequences of which are given in Table I below, were obtained from EPYTOP (Nimes, France). The purity (>70%) is controlled by reverse-phase high performance liquid chromatography (HPLC). The peptides are lyophilized and then dissolved in DMSO at 10 mg/ml, and stored at −80° C.

The response of the various TIL clones was evaluated, by means of a TNF-release assay, after 6 hours of co-culturing with EBV cells expressing an HLA-B*3501 molecule loaded with 10 μM of the peptide of interest. The results are combined in Table I.

TABLE 1

| | |
|---|---|
| Position of | TNF |

| | | |
|---|---|---|
| the peptides | Sequence | pg/ml |

| | | |
|---|---|---|
| Melan-A | | |
| 22-33 | TTAEEAAGIGIL (SEQ ID NO: 21) | 2 |
| 23-35 | TAEEAAGIGILTV (SEQ ID NO: 6) | 107 |
| 26-37 | EAAGIGILTVIL (SEQ ID NO: 7) | 125 |
| 26-35 | EAAGIGILTV (SEQ ID NO: 8) | 182 |
| 27-35 | AAGIGILTV (SEQ ID NO: 22) | 4 |
| 26-34 | EAAGIGILT (SEQ ID NO: 23) | 13 |
| gp100 | | |
| 470-479 | QVPLDCVLYR (SEQ D NO: 24) | 430 |
| 471-479 | VPLDCVLYR (SEQ ID NO: 3) | 170 |
| 471-480 | VPLDCVLYRY (SEQ ID NO: 13) | 380 |
| 472-480 | PLDCVLYRY (SEQ ID NO: 20) | 2.8 |
| Tyrosinase | | |
| 309-322 | TPRLPSSADVEFCL (SEQ ID NO: 14) | 308 |
| 309-321 | TPRLPSSADVEFC (SEQ ID NO: 25) | 214 |
| 309-320 | TPRLPSSADVEF (SEQ ID NO: 4) | 605 |
| 309-319 | TPRLPSSADVE (SEQ ID NO: 26) | 89 |
| 310-322 | PRLPSSADVEFCL (SEQ ID NO: 27) | 27 |
| 309-318 | TPRLPSSADV (SEQ ID NO: 28) | 0.2 |
| 312-320 | LPSSADVEF (SEQ ID NO: 29) | 7.4 |
| 312-321 | LPSSADVEFC (SEQ ID NO: 30) | 9.7 |
| MAGE-A3 | | |
| 168-176 | EVDPIGHLY (SEQ ID NO: 19) | 265 |
| MAGE-A6 | | |
| 168-176 | EVDPIGHVY (SEQ ID NO: 2) | 43 |
| NY-ESO-1 | | |
| 92-104 | LAMPFATPMEAEL (SEQ ID NO: 15) | 345 |
| 92-103 | LAMPFATPMEAE (SEQ ID NO: 16) | 219 |
| 94-104 | MPFATPMEAEL (SEQ ID NO: 17) | 264 |
| 94-103 | MPFATPMEAE (SEQ ID NO: 18) | 292 |
| 94-102 | MPFATPMEA (SEQ ID NO: 5) | 258 |
| 92-100 | LAMPFATPM (SEQ ID NO: 31) | 87 |
| 96-104 | FATPMEAEL (SEQ ID NO: 32) | 0 |

In addition, the antigenicity of the peptides was tested by evaluating the ability of the TIL clones to lyse BM36.1 cells (KELLY et al., 1992, Nature, 355, 641-4) which are deficient in TAP transporter, and which naturally express HLA-B*3501 and HLA-A*0101. The BM36.1 cells are labeled for 1 h at 37° C. with 100 Ci of ⁵¹Cr (Na2⁵¹CrO4, ORIS, Gif-sur-Yvette, France). The cells are then pulsed for 20 minutes with the various synthetic peptides. 10³ BM36.1 cells thus treated are incubated with 10⁴ T cells of the clone (effector:target ratio of 10:1) for 4 hours. The culture supernatants are recovered and the amount of ⁵¹Cr released is evaluated by means of β plate counter (EG&G Wallac, Evry, France). For each TIL clone, a negative control is carried out with a non-relevant peptide.

The amount of peptide necessary to obtain 50% of the maximum lysis (EC50) was determined. In addition, the affinity and the stability of the peptides for HLA-B35 were measured as described by TOURDOT et al., Eur. J. Immunol., 30:3411-3421, 2000). To measure the affinity of the peptides, the BM36.1 cells were incubated for 18 h with a range of concentrations of each peptide. The BM36.1 cells were incubated in parallel with a range of a reference peptide which binds to HLA-B35 (peptide 37F, TAKAMIYA et al., Int. Immunol., 6: 255-261, 1994; SCHÖNBACH et al., J. Immunol., 154: 5951-5958, 1995). The relative amount of peptide bound is then estimated at each concentration, for each of the peptides, by measuring HLA-B35 stabilization at the cell surface. This measurement is carried out by flow cytometry using an anti-HLA-B/C antibody (which recognizes, in the case of BM36.1 cells, only HLA-B*3501, these cells spontaneously expressing no HLA-C molecule). The % of binding to B*35 is then calculated for each concentration, by fixing the 100% binding for 100 μM for each peptide. The relative affinity (RA) is then determined in the following way.

RA=[concentration of peptide] for 20% binding/[concentration of the peptide 37F] for 20% binding.

To measure the stability of the peptides, the BM36.1 cells are incubated for 18 h with 100 μM of each of the peptides. They are then incubated in the presence of BFA (10 μg/ml) for one hour in order to block the transport of newly synthesized HLA molecules to the cell surface. The BM36.1 cells are washed in PBS and taken up in culture medium containing 5% SVF and 0.5 μg/ml of BFA, which constitutes the time 0. Cells are then removed after 30 minutes, 1 h, 2 h, 4 h and 6 h of incubation. The relative amount of peptide bound is then estimated at each time, for each of the peptides, by measuring HLA-B35 stabilization at the cell surface. This measurement is carried out by flow cytometry using an anti HLA-B/C antibody. The time given in Table II corresponds to the half-life time of the peptide on the HLA-B*35.

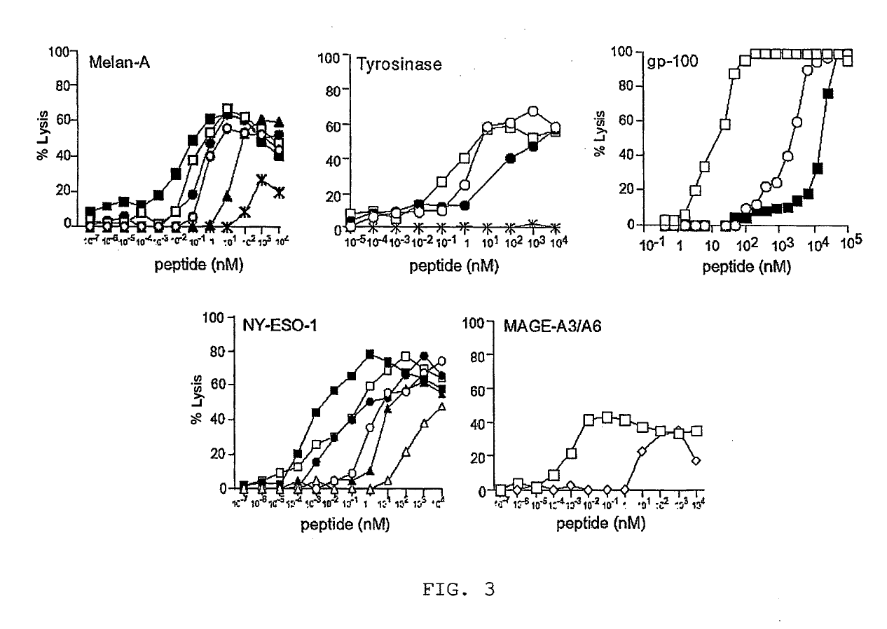

The results are given in FIG. 3, and Table II below.

TABLE II

Position of

| | | | | |
|---|---|---|---|---|
| the peptides | Sequence | EC 50 | RA | Stability |

| | | | | |
|---|---|---|---|---|
| Mefan-A | | | | |
| 26-35 | EAAGIGILTV (●; SEQ ID NO: 8) | 0.5 nM | 5.9 | 1h30 |
| 26-35 | EAAGIGILTY (▪; SEQ ID NO: 9) | 0.01 nM | 0.75 | >6h |
| 26-34 | EAAGIGILY (▴; SEQ ID NO: 10) | 10 nM | 1.32 | >6h |
| 26-35 | EPAGIGILTY □; SEQ ID NO: 11) | 0.05 nM | 0.66 | >6h |
| 26-34 | EPAGIGILY (*; SEQ ID NO: 33) | 100 nM | 0.4 | >6h |
| 26-35 | EPAGIGILTV (◯; SEQ ID NO: 12) | 0.8 nM | 5.1 | 1h30 |
| gp100 | | | | |
| 471-480 | VPLDCVLYRY (□; SEQ ID NO: 13) | 0.02 nM | 2.6 | >6h |
| 471-479 | VPLDCVLYR (◯; SEQ ID NO: 3) | 2 nM | 20 | <30 min |
| 470-479 | QVPLDCVLYR (▪; SEQ ID NO: 24) | 20 μM | — | — |
| Tyrosinase | | | | |
| 309-322 | TPRLPSSADVEFCL (◯; SEQ ID NO: 14) | 2 nM | >20 | >6h |
| 309-321 | TPRLPSSADVEFC (●; SEQ ID NO: 25) | 12 nM | 16.7 | <30 min |
| 309-320 | TPRLPSSADVEF □; SEQ ID NO: 4) | 0.2 nM | 1.3 | >6h |
| 312-320 | LPSSADVEF (*; SEQ ID NO: 29) | — | 1.25 | 6h |
| MAGE-A3 | | | | |
| 168-176 | EVDPIGHLY (◯; SEQ ID NO: 19) | 0.45 pM | 2.3 | >6h |
| MAGE-A6 | | | | |
| 168-176 | EVDPIGHVY (⋄; SEQ ID NO: 2) | 4.2 nM | 1.1 | 6h |
| NY-ESO-1 | | | | |
| 92-104 | LAMPFATPMEAEL (□; SEQ ID NO: 15) | 0.1 nM | 0.58 | >6h |
| 92-103 | LAMPFATPMEAE (▴; SEQ ID NO: 16) | 5 nM | 0.75 | >6h |
| 94-104 | MPFATPMEAEL (●; SEQ ID NO: 17) | 0.05 nM | 1.32 | >6h |
| 94-103 | MPFATPMEAE (◯; SEQ ID NO: 18) | 1 nM | 0.66 | >6h |
| 94-102 | MPFATPMEA (▪; SEQ ID NO: 5) | 1 pM | 0.4 | >6h |
| 92-100 | LAMPFATPM (Δ; SEQ ID NO: 31) | 100 nM | 5.1 | 1h30 |

Legend of FIG. 3: Along the y-axis, the percentage of cell lysis obtained. Along the x-axis, the concentration of peptide (in nM).

The results in Tables I and II and in FIG. 3 show that:

* Three overlapping Melan-A peptides are recognized by the clone M28.10B. The peptide most effectively recognized is the decapeptide 26-35 (EAAGIGILTV, SEQ ID NO: 8). This Melan-A peptide 26-35 probably corresponds to the peptide naturally presented, in the B*3501 context, by the melanoma cells and recognized by the TILs from patient M28. It is known that this same Melan-A peptide 26-35 is presented by HLA molecules, but only in the HLA-A*0201 context (KAWAKAMI et al., 1994, J. Exp. Med, 180, 347-52; VALMORI et al., 1998, J Immunol, 160, 1750-8). Cross presentation of the same epitope by various isotypes of HLA class I molecules is relatively uncommon.

* The gp100-specific clones (M28.9B) recognizes, in the HLA-B*3501 context, three overlapping peptides (two decamers and one nonamer) located between amino acids 470 and 480. However, high concentrations of the peptide QVPLDCVLYR (SEQ ID NO: 24) are required to induce a response of the specific T clone (EC50 at 20 μM). The affinity and stability measurement results for this peptide show that it is not capable of binding to HLA B*35. The weak response obtained by the clone is explained by contamination of this peptide (prepared at 70% purity) with other peptides recognized by this T clone.

Several gp100-derived epitopes presented in the HLA-A or HLA-C contexts are known (CASTELLI et al., 1999, J Immunol, 162, 1739-48; KAWAKAMI et al., 1998; TSAI et al., 1997, J Immunol, 158, 1796-802). On the other hand, gp100 epitopes presented in the HLA-B context were not, until now, known.

* The tyrosinase-specific clone (M171.100B) effectively recognizes three overlapping peptides: the 14-mer 309-322, the 13-mer 309-321, and the 12-mer 309-320. The 12-mer is the peptide most effectively recognized with 50% of the maximum lysis obtained at 0.2 nM, versus 2 nM and 12 nM for the 14-mer and the 13-mer, respectively (FIG. 3). The deletion of the phenylalanine at the C-terminal end of the 12-mer greatly reduces the recognition by the CTLs. The peptide 312-320, which has been shown to be recognized by a CTL clone in the HLA-B*3501 context (MOREL et al., 1999, Int J Cancer, 83 755-9) is not recognized by the TIL clone M171.100B. In addition, while examples of epitopes of cells of 11-mer type (AARNOUDSE et al., 1999, Int. J Cancer, 82, 442-8; CHIARI et al., 1999, Cancer Res, 59, 5785-92; KAWAKAMI et al., 2001, J Immunol, 166, 2871-7) and 14-mer type exist (PROBST-KEPPER et al., 2001, J Exp Med, 193, 1189-98), they are epitopes which are significantly larger than the epitopes usually presented by T cells.

*The MAGE-A3/A6-specific clone M171.8C recognizes the MAGE-A3/B*3501 epitope EVDPIGHLY previously described by SCHULTZ et al., (2001, mentioned above). As this TIL clone also reacts with MAGE-A6, this indicates that the MAGE-A6 peptide 168-176 (EVDPIGHVY), which differs from the MAGE-A3 epitope by virtue of a single amino acid at position 8, constitutes another HLA-B*3501-restricted melanoma epitope.

*The clone specific for an NY-ESO-1 epitope in the B*3501/B*3503 contexts (M118.45) effectively recognizes five overlapping peptides: the 13-mer 92-104, the 12-mer 92-103, the 11-mer 94-104, the decamer 94-103 and the nonamer 94-102. The nonamer 94-102 is the peptide most effectively recognized, with 50% of the maximum lysis obtained at 1 pM, versus 0.05 nM for the 11-mer, 0.1 nM for the 13-mer, 1 nM for the decamer and 5 nM for the 12-mer.

It is known that the nonamers 94-102 and 92-100 are presented in the HLA-B51 (JAGER et al, Cancer Immunity, 2002, vol 2, page 12) and HLA-Cw3 (GNJATIC et al., 2000, Proc Natl Acad Sci USA, 97, 10917-22) contexts, respectively. However, their presentation in an HLA-B35 context had not previously been described.

### EXAMPLE 4

### Modification of the Antigenic Peptides Presented in the HLA-B35 Context

Some of the antigenic peptides identified do not have the appropriate anchoring residues for HLA-B*3501 molecules, i.e. P, A or V at position 2, Y, F, M, L or I at position 9, for the nonamers, and at position 10 for the decamers (FALK et al., 1993, Immunogenetics, 38, 161-2 [erratum in Immunogenetics 1994; 39(5):379]).

In order to attempt to increase the affinity of this peptide for the HLA-B*3501 context and the T cell response, the inventors introduced modified residues at positions 2 and/or 9, or 10, of the Melan-a/MART1 peptides 26-34 and 26-35, respectively.

The results obtained with these modified peptides are given in FIG. 3 (Melan A). It is observed that the modified decapeptides EAAGIGILTY (SEQ ID NO: 9) and EPAGIGILTY (SEQ ID NO: 11) induce maximum lysis by the clone M28.10B at concentrations 10 to 50 times lower than the wild-type peptide (10 and 50 pM instead of 500 pM). In addition, these peptides exhibit improved stability (FIG. 3).

These modifications therefore make it possible to increase the binding affinity of these peptides for HLA-B*3501, but also to induce greater reactivity of the specific TIL clone.

### EXAMPLE 5

### Natural Presentation of the Antigenic Peptides in the HLA-B35 Context by Melanoma Cells

To test the value, as targets for immunotherapy, of the various antigenic peptides identified in examples 3 and 4, the inventors analyzed their presentation by a panel of melanoma cell lines expressing the various antigens, from which these peptides are derived, and HLA-B*3501 molecules. To increase the cell surface expression of the HLA molecules, the melanoma cells were preincubated, for some experiments, for 48 hours in medium containing 500 U/ml of γ-IFN (Tebu, Paris, France).

The response of the various TIL clones with respect to these lines is detected by measuring their TNF production as in example 1 (effector:target ratio of 1:3).

Negative controls were carried out using melanoma cell lines not expressing HLA molecules (M113).

The melanoma cell lines were established from fragments of metastatic tumors or tumors which had invaded the lymph nodes, and placing culture in RPMI 1640 medium (Sigma, St. Louis, USA) containing 10% of fetal calf serum (Gibco-BRL, Cergy-Pontoise, France), penicillin (10 mg/ml), streptomycin (10 U/ml) (Sigma) and L-glutamine (2 nM) (Sigma, St. Louis, USA).

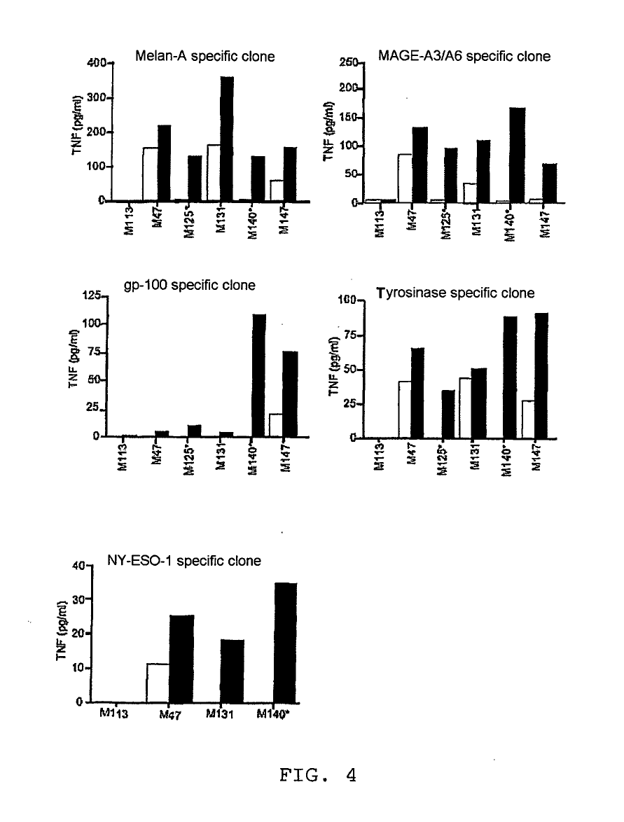

The results are given in FIG. 4: along the y-axis, the TNF concentration in pg/ml; along the x-axis, the melanoma lines expressing HLA-B35 and the various antigens (M47, M125, M131, M140 and M147), or not expressing the HLA-B35 allele (M113).

▪: melanoma cell lines pretreated with 500 U/ml of γ-IFN.

□: melanoma cell lines not pretreated with γ-IFN.

The clones specific for Melan-A, for tyrosinase and for MAGE-A3 recognize the melanoma cell lines M47, M131 and M147 independently of a treatment with γ-IFN. However, these same clones recognize the M125 and M140 lines only after induction of the expression of HLA-B35 by treatment with γ-IFN.

The gp100 specific clone recognizes a melanoma line independently of treatment with y-IFN (M147). This clone also recognizes the M125 and M140 lines after treatment with γ-IFN (weakly for M125).

The NY-ESO-1-specific clone recognizes one of these lines spontaneously expressing this antigen (M47) and the other two lines after treatment with γ-IFN (M131 and M140, FIG. 4).

In the case of the M125 and M140 lines, the lack of recognition of these lines by the various TIL clones is probably related to a limited number of MHC/peptide complexes present at the surface of these cells. In fact, these two cell lines express the HLA-B35 molecule only after treatment with γ-IFN.

The spontaneous recognition of the melanoma lines by the various CD8 T clones shows that the epitopes identified are naturally presented by these tumors.

### EXAMPLE 6

### Antigenicity of the Mutate ras Peptide 55-64^{Q61R}

The wild-type ras peptides 55-64^{WT} (ILDTAGQEEY; SEQ ID NO: 34), the mutated decamer 55-64^{Q61R}, and the MAGE-A3 peptide (EVDPIGHLY; SEQ ID NO: 20) were obtained from SYNT:EM (Nimes, France). The purity (>85%) is controlled by reverse-phase high performance liquid chromatography. The peptides are lyophilized and then dissolved in DMSO at 10 mg/ml, and stored at −80° C.

The antigenicity of the peptide 55-64^{Q61R} and that of its wild-type analog (55-64^{WT}) are evaluated by testing the ability of these peptides to induce the growth of specific cytotoxic T lymphocytes (CTLs) by in vitro stimulation of peripheral blood mononuclear cells (PBMCs) with dendritic cells (DCs) pulsed with these peptides.

The CD8+ lymphocytes are obtained from the PBMCs of an HLA-A*0101 donor by negative sorting of the CD4+ T cells on magnetic beads (MILTENY BIOTECH, France).

The dendritic cells are prepared from adherent PBMCs placed in culture for 7 days in 6-well culture plates containing RPMI culture medium supplemented with 10% fetal calf serum, 50 ng/ml of GM-CSF (SIGMA) and 50 ng/ml of IL-4 (SIGMA). On d+7, maturation of the dendritic cells is induced for 2 days in an RPMI culture medium supplemented with 10% fetal calf serum, 10 ng/ml of TNF-α (SIGMA) and 100 μg/ml of poly-IC (SIGMA). On d+9, the matured dendritic cells are incubated for 2 hours with 5 μg/ml of ras peptide 55-64^{Q61R} or of ras peptide 55-64^{WT}; they are then washed in order to remove the free peptides.

The dendritic cells pulsed with the ras peptide 55-64^{Q61R} or the ras peptide 55-64^{WT} are used to stimulate the CD8+ lymphocytes (3×10⁷ cells). 3 stimulations are performed at one-week intervals.

Each culture well is tested for the presence of CTLs specific for the peptide.

For this purpose, 7 days after the final stimulation, 2×10⁶ BM36.1 cells expressing HLA-A*0101 (KELLY et al., Nature. 355. 641. 1992) incubated at 37° C. for 12 hours in RPMI supplemented with 100 μM of N-ras peptide 55-64 WT or 55-64^{Q61R}, and 1 μM of β2-microglobulin, and washed in PBS, are added to each well.

The specific CTL response to the stimulation with the wild-type or mutated N-ras peptide is measured by assaying γ-interferon (γ-IFN), as described by LABARRIERE et al. (Int. J. Cancer, 78, 209, 1998).

Two of the five culture wells stimulated with the ras peptide exhibit a CTL proliferation specific for the peptide (0.3 and 0.5% of reactive cells per well), whereas no proliferation is observed in the wells stimulated with the peptide 55-64^{WT}.

### EXAMPLE 7

### Properties of a T Lymphocyte Clone Induced with the Peptide 55-64^{Q61R}

Lymphocyte clones were obtained by limiting dilution from the cells of the culture wells containing 0.5% of reactive T cells.

The ability of the T cells derived from one of these clones to lyse BM36.1 cells presenting the peptide 55-64^{Q61R} or the peptide 55-64^{WT} is evaluated according to a standard ⁵¹Cr-release assay (HERIN et al., Int. J.

Cancer, 39, 390-396, 1987).

BM36.1 cells are labeled with ⁵¹Cr (Na2⁵¹CrO4, ORIS, Gif-sur-Yvette, France). The cells are then pulsed for 1 hour at 37° C. with 10 μM of peptide 55-64^{WT} or of peptide 55-64^{Q61R}, and washed. 10³ BM36.1 cells thus treated are incubated with 5×10³ T cells of the clone to be tested, for 4 hours. The culture supernatants are recovered and the percentage of released ⁵¹Cr is evaluated.

Considerable lysis of the BM36.1 cells pulsed with the mutated ras peptide 55-64^{Q61R} is observed. On the other hand, the lysis of the BM36.1 cells pulsed with the wild-type ras peptide is very low.

The specificity of this clone with respect to cells expressing HLA-A*0101 and the wild-type protein or the N-ras protein bearing the Q61R mutation is evaluated on transfected COS cells or on HLA-A*0101 melanoma cells, expressing or not expressing the Q61R mutation.

For the transfection of the COS cells, the 334 pb cDNA encoding a fragment of the wild-type N-ras protein is obtained by PCR amplification from a complete cDNA of the wild-type N-ras protein. A cDNA encoding a fragment of the N-ras protein mutated at position 61 by substitution of the glutamine with an arginine is obtained by site-directed mutagenesis.

The wild-type or mutated cDNA is inserted into the vector pcDNA3 and amplified in the E. coli bacterial strain TOP 10 F′ (INVITROGEN, reference C2020-03).

A cDNA encoding the HLA-A*0101 molecule is introduced into the vector pcDNA 3.1 (INVITROGEN, reference CV790-20).

COS-7 cells are cotransfected with these constructs as described below:

COS-7 cells (BRICHARD et al., J. Exp. Med., 178, 489, 1993) are cultured in DMEM medium (BIOWHITTAKER) containing 10% fetal calf serum, 100 U/ml penicillin, 100 μg/ml of streptomycin (SIGMA, St Louis, USA) and 2 mM of L-glutamine (SIGMA, St Louis, USA).

16.5×10³ COS cells are cotransfected with 100 ng of a mixture of the vector pcDNA 3.1 expressing HLA-A*0101 and of a vector pcDNA3 expressing the wild-type or mutated N-ras protein, by the chloroquin-dextran-DEAE method (BRICHARD et al., J. Exp. Med., 178, 489, 1993; SEED et al., PNAS, 84, 3365, 1987). The details of this method are described by DE PLAEN et al., (Methods, 12, 125, 1997).

The T cell stimulation is measured by assaying the TNF (DE PLAEN et al., Methods, 12, 125, 1997; LABARRIERE et al., Int. J. Cancer, 78, 209, 1998).

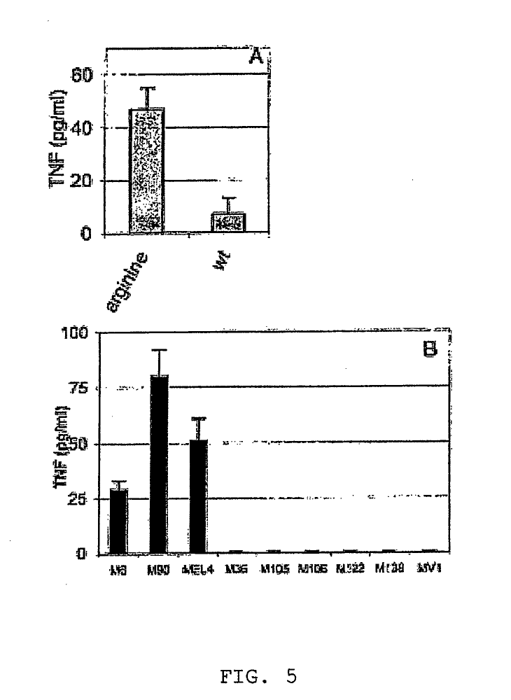

2×10³ to 10⁴ cells of the T clone tested are added to 3×10⁴ COS cells, 48 hours after transfection, or to 3×10⁴ melanoma cells. The culture supernatants are recovered 6 hours later and their TNF content is determined by measuring their cytotoxic effect on the WEHI 164 murine fibrosarcoma clone 13 (BRICHARD et al., J. Exp. Med., 178, 489, 1993) by MTT calorimetric assay. The results obtained with the transfected COS cells are shown in FIG. 5a: along the x-axis, wild-type ras peptides or ras peptides mutated at position 61 by substitution of the glutamine with an arginine; along the y-axis, TNF concentration in pg/ml.

The results show that the T cells of the clone are strongly stimulated by the cells expressing the Q61R N-ras protein, whereas only a weak stimulation is observed with the wild-type N-ras protein.

The results obtained with the melanoma cells are shown in FIG. 5b: along the x-axis, melanoma cell lines expressing the ras mutation Q61R (M6, M90, MEL4) or not expressing it (M36, M105, M106, M122, M138, MV1); along the y-axis, TNF concentration in pg/ml.

These results show that, among the HLA-A*0101 melanoma lines, only those expressing the Q61R mutation (lines M6, M90 and MEL4) stimulate a response of the T cells of the clone.

### EXAMPLE 8

### Identification of an Antigenic Peptide Presented in the HLA-A*0101 Context

A CD8+ T clone, derived from a population of melanoma TILs, recognizes an undescribed peptide derived from the gp100 antigen, in the A*0101 context (PLDCVLYRY, SEQ ID NO: 20). The antigenicity of this peptide was tested by evaluating the ability of the clone to lyse BM36.1 cells presenting the peptide of interest in the HLA-A*0101 context. The BM36.1 cells are labeled for 1 h at 37° C. with 100 Ci of ⁵¹Cr (Na2⁵¹CrO4, ORIS, Gif-sur-Yvette, France). The cells are then pulsed for 20 minutes with the various synthetic peptides. 103 BM36.1 cells thus treated are incubated with 104 T cells of the clone (effector:target ratio of 10:1) for 4 hours. The culture supernatants are recovered and the amount of ⁵¹Cr released is evaluated by means of a P plate counter (EG&G Wallac, Evry, France). A negative control is carried out with a non-relevant peptide.

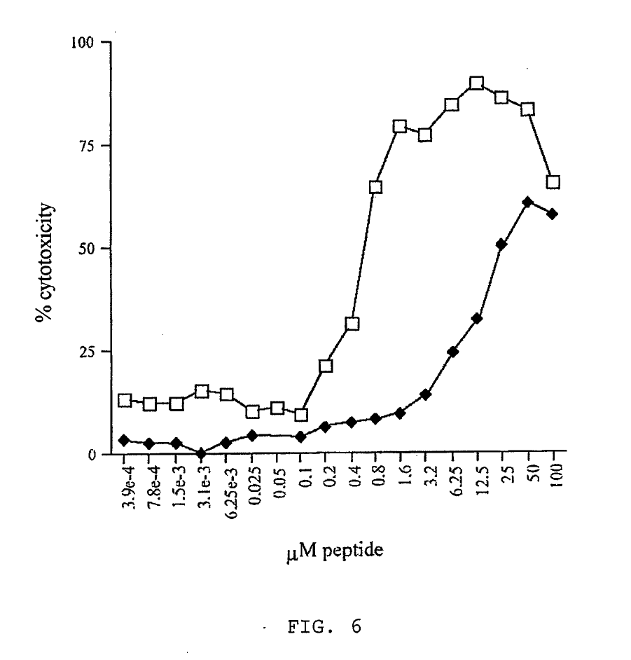

The results are given in FIG. 6: along the y-axis, the percentage of cell lysis obtained; along the x-axis, the concentration of peptide PLDCVLYRY (□), or VPLDCVLYRY (♦). The EC50 for the peptide PLDCVLYRY is 0.6 μM, whereas the EC50 for the peptide PLDCVLYRY is 10 μm.

The sequence of this peptide is included in the sequence of the decamer recognized in the B*3501 context, but this peptide is not recognized by the clone M28.9B. On the other hand, the peptide VPLDCVLYRY (SEQ ID NO: 13) is also recognized in the A*0101 context by the clone M199.6.12 (Table I and FIG. 6).

In addition, the spontaneous recognition, by the clone M199.6.12, of melanoma lines sharing HLA-A*0101 shows that this epitope is effectively presented by these tumors.

1. A medicinal product for antitumor immunotherapy in an HLA-B35 patient comprising at least one immunogenic peptide representing a T epitope presented by MHC I, selected from the group consisting of:
a) a peptide comprising the sequence EX₁AGIGILX₂ (SEQ ID NO: 1) in which X₁ represents A or P, and X₂ represents T or Y, capable of inducing a cytotoxic response directed against the Melan-A antigen;
b) a peptide comprising the sequence EVDPIGHVY (SEQ ID NO: 2), capable of inducing a cytotoxic T response directed against the MAGE-A6 antigen;
c) a peptide comprising the sequence VPLDCVLYR (SEQ ID NO: 3), capable of inducing a cytotoxic response directed against the gp 100 antigen;
d) a peptide comprising the sequence TPRLPSSADVEF (SEQ ID NO: 4), capable of inducing a cytotoxic response directed against the tyrosinase antigen; and
e) a peptide comprising the sequence MPFATPMEA (SEQ ID NO: 5), capable of inducing a cytotoxic response directed against the NY-ESO-1 antigen.

2. The medicinal product of claim 1, wherein said peptide is selected from the group consisting of:
a) a peptide of sequence selected from the group consisting of TAEEAAGIGILTV (SEQ ID NO: 6), EAAGIGILTVIL (SEQ ID NO: 7), EAAGIGILTV (SEQ ID NO: 8), EAAGIGILTY (SEQ ID NO: 9), EAAGIGILY (SEQ ID NO:10), EPAGIGILTY (SEQ ID NO:11), and EPAGIGILTV (SEQ ID NO: 12);
b) a peptide of sequence EVDPIGHVY (SEQ ID NO: 2);
c) a peptide of sequence selected from the group consisting of VPLDCVLYR (SEQ ID NO: 3) and VPLDCVLYRY (SEQ ID NO: 13);
d) a peptide of sequence selected from the group consisting of TPRLPSSADVEFCL (SEQ ID NO: 15) and TPRLPSSADVEF (SEQ ID NO: 4); and
e) a peptide of sequence selected from the group consisting of LAMPFATPMEAEL (SEQ ID NO: 16), LAMPFATPMEAE (SEQ ID NO:17), MPFATPMEAEL (SEQ ID NO: 18), MPFATPMEAE (SEQ ID NO:19) and MPFATPMEA (SEQ ID NO: 5).

3. An immunogenic peptide representing a T epitope presented by MHC I, selected from the group consisting of:
a peptide of sequence selected from the group consisting of EAAGIGILTY (SEQ ID NO: 9), EAAGIGILY (SEQ ID NO:10), EPAGIGILTY (SEQ ID NO:11), and EPAGIGILTV (SEQ ID NO: 12);
a peptide of sequence selected from the group consisting of VPLDCVLYR (SEQ ID NO: 3), VPLDCVLYRY (SEQ ID NO: 13) and PVPLDCVLYRY (SEQ ID NO: 14); and
a peptide of sequence selected from the group consisting of TPRLPSSADVERFCL (SEQ ID NO: 15) and TPRLPSSADVEF (SEQ ID NO: 4).

4. A multiepitope composition comprising at least two peptides of two different categories among the categories a), b), c), d) and e) as defined in claim 1.

5. A multiepitope composition comprising at least one peptide from each of categories a), b), c), d) and e) as defined in claim 1.

6. The multiepitope composition as claimed in claim 4, consisting of a chimeric polypeptide comprising one or more copies of each of said peptides.

7. A polynucleotide encoding a chimeric polypeptide as claimed in claim 6.

8. An antigen-presenting cell expressing an MHC I HLA-B35 allele, wherein said cell expresses a peptide as defined in claim 1.

9. An antigen-presenting cell expressing an MHC I HLA-B35 allele, wherein said cell is transfected with a polynucleotide as claimed in claim 7.

10. A method for in vitro detection of CTLs directed against one or more of antigens selected from the group consisting of Melan-A, MAGE-A6, gp100, tyrosinase and NY-ESO-1, comprising
obtaining a biological sample from an HLA-B35 individual;
contacting said biological sample with at least one peptide defined in claim 1; and detecting the presence or absence of a CTL directed against one or more of the antigens selected from the group consisting of Melan-A MAGE-A6, gp100, tyrosinase and NY-ESO-1.

11. The multiepitope composition as claimed in claim 5, consisting of a chimeric polypeptide comprising one or more copies of each of said peptides.

12. A polynucleotide encoding a chimeric polypeptide as claimed in claim 11.

13. An antigen-presenting cell expressing an MHC I HLA-B35 allele, wherein said cell is transfected with a polynucleotide as claimed in claim 12.

14. A multiepitope composition comprising at least two peptides of two different categories among the categories a), b), c), d) and e) as defined in claim 2.

15. The multiepitope composition as claimed in claim 14, consisting of a chimeric polypeptide comprising one or more copies of each of said peptides.

16. A polynucleotide encoding a chimeric polypeptide as claimed in claim 15.

17. An antigen-presenting cell expressing an MHC I HLA-B35 allele, wherein said cell is transfected with a polynucleotide as claimed in claim 16.

18. A multiepitope composition comprising at least one peptide from each of categories a), b), c), d) and e) as defined in claim 2.

19. The multiepitope composition as claimed in claim 18, consisting of a chimeric polypeptide comprising one or more copies of each of said peptides.

20. A polynucleotide encoding a chimeric polypeptide as claimed in claim 19.

21. An antigen-presenting cell expressing an MHC I HLA-B35 allele, wherein said cell is transfected with a polynucleotide as claimed in claim 20.

22. An antigen-presenting cell expressing an MHC I HLA-B35 allele, wherein said cell expresses a peptide as defined in claim 2.

23. A method for in vitro detection of CTLs directed against one or more of antigens selected from the group consisting of Melan-A, MAGE-A6, gp 100, tyrosinase and NY-ESO-1, comprising
obtaining a biological sample from an HLA-B35 individual;
contacting said biological sample with at least one peptide defined in claim 2; and
detecting the presence or absence of a CTL directed against one or more of the antigens selected from the group consisting of Melan-A, MAGE-A6, gp100, tyrosinase and NY-ESO-1.

24. A method for in vitro detection of CTLs directed against one or more of antigens selected from the group consisting of Melan-A, MAGE-A6, gp100, tyrosinase and NY-ESO-1, comprising
obtaining a biological sample from an HLA-B35 individual;
contacting said biological sample with at least one peptide defined in claim 3; and
detecting the presence or absence of a CTL directed against one or more of the antigens selected from the group consisting of Melan-A, MAGE-A6, gp100, tyrosinase and NY-ESO-1.


SEQUENCE LISTING (35 sequences)

SEQ ID NO: 1 - protein, 9 aa, Artificial Sequence
  Synthetic Peptide
EXAGIGILX

SEQ ID NO: 2 - protein, 9 aa, Homo sapiens
EVDPIGHVY

SEQ ID NO: 3 - protein, 9 aa, Homo sapiens
VPLDCVLYR

SEQ ID NO: 4 - protein, 12 aa, Homo sapiens
TPRLPSSADVEF

SEQ ID NO: 5 - protein, 9 aa, Homo sapiens
MPFATPMEA

SEQ ID NO: 6 - protein, 13 aa, Homo sapiens
TAEEAAGIGILTV

SEQ ID NO: 7 - protein, 12 aa, Homo sapiens
EAAGIGILTVIL

SEQ ID NO: 8 - protein, 10 aa, Homo sapiens
EAAGIGILTV

SEQ ID NO: 9 - protein, 10 aa, Artificial Sequence
  Synthetic Peptide
EAAGIGILTY

SEQ ID NO: 10 - protein, 9 aa, Artificial Sequence
  Synthetic Peptide
EAAGIGILY

SEQ ID NO: 11 - protein, 10 aa, Artificial Sequence
  Synthetic Peptide
EPAGIGILTY

SEQ ID NO: 12 - protein, 10 aa, Artificial Sequence
  Synthetic Peptide
EPAGIGILTV

SEQ ID NO: 13 - protein, 10 aa, Homo sapiens
VPLDCVLYRY

SEQ ID NO: 14 - protein, 14 aa, Homo sapiens
TPRLPSSADVEFCL

SEQ ID NO: 15 - protein, 13 aa, Homo sapiens
LAMPFATPMEAEL

SEQ ID NO: 16 - protein, 12 aa, Homo sapiens
LAMPFATPMEAE

SEQ ID NO: 17 - protein, 11 aa, Homo sapiens
MPFATPMEAEL

SEQ ID NO: 18 - protein, 10 aa, Homo sapiens
MPFATPMEAE

SEQ ID NO: 19 - protein, 9 aa, Homo sapiens
EVDPIGHLY

SEQ ID NO: 20 - protein, 9 aa, Homo sapiens
PLDCVLYRY

SEQ ID NO: 21 - protein, 12 aa, Homo sapiens
TTAEEAAGIGIL

SEQ ID NO: 22 - protein, 9 aa, Homo sapiens
AAGIGILTV

SEQ ID NO: 23 - protein, 9 aa, Homo sapiens
EAAGIGILT

SEQ ID NO: 24 - protein, 10 aa, Homo sapiens
QVPLDCVLYR

SEQ ID NO: 25 - protein, 13 aa, Homo sapiens
TPRLPSSADVEFC

SEQ ID NO: 26 - protein, 11 aa, Homo sapiens
TPRLPSSADVE

SEQ ID NO: 27 - protein, 13 aa, Homo sapiens
PRLPSSADVEFCL

SEQ ID NO: 28 - protein, 10 aa, Homo sapiens
TPRLPSSADV

SEQ ID NO: 29 - protein, 9 aa, Homo sapiens
LPSSADVEF

SEQ ID NO: 30 - protein, 10 aa, Homo sapiens
LPSSADVEFC

SEQ ID NO: 31 - protein, 9 aa, Homo sapiens
LAMPFATPM

SEQ ID NO: 32 - protein, 9 aa, Homo sapiens
FATPMEAEL

SEQ ID NO: 33 - protein, 9 aa, Artificial Sequence
  Synthetic Peptide
EPAGIGILY

SEQ ID NO: 34 - protein, 10 aa, Homo sapiens
ILDTAGQEEY

SEQ ID NO: 35 - protein, 10 aa, Homo sapiens
ILDTAGREEY